(19) United States
(12) Patent Application Publication
(10) Pub. No.: US 2025/0161195 A1
(43) Pub. Date: May 22, 2025

(54) METHOD FOR OBTAINING PLANT EXTRACTS COMPRISING AN AUTOFERMENTATION STEP, COMPOSITIONS COMPRISING SUCH EXTRACTS AND COMETIC USES THEREOF

(71) Applicant: ISP INVESTMENTS LLC, Wilmington, DE (US)

(72) Inventors: Elodie OGER, ANTIBES (FR); Laurine BERGERON, ANTIBES (FR); Laura MOURET, BIOT (FR); Florian LABARRADE, ANTIBES (FR); Jean-Marie Botto, VALBONNE (FR); Yolène FERREIRA, GRASSE (FR); Marie BRULAS, VILLENEUVE-LOUBET (FR)

(73) Assignee: ISP INVESTMENTS LLC, Wilmington, DE (US)

(21) Appl. No.: 18/836,321

(22) PCT Filed: Feb. 6, 2023
(86) PCT No.: PCT/EP2023/052799

§ 371 Date: Aug. 6, 2024

(30) Foreign Application Priority Data
Feb. 8, 2022 (FR) FR2201093

(51) Int. Cl. A61K 8/9789; A61Q 19/08; C12P 7/42

(52) U.S. Cl. CPC A61K 8/9789; A61Q 19/08; C12P 7/42

(57) ABSTRACT

The invention relates to processes for preparing plant extracts comprising an autofermentation step and optionally extraction steps allowing enrichment of the extracts with small molecular weight RNA. The invention also relates to compositions comprising such extracts and their cosmetic uses to combat the signs of skin aging. loss of firmness or tone. improve barrier function and hydration. lighten the skin or improve the skin's innate immune defenses and well-being in vivo.

### TECHNICAL FIELD

The present application relates to the field of cosmetics and more particularly to the preparation of active extracts of plant origin used in cosmetic formulations to fight against the signs of skin aging, loss of firmness, improve barrier function and hydration or lighten the skin.

### TECHNICAL BACKGROUND OF THE INVENTION

The preparation of plant extracts usable in cosmetics using all the purification techniques known in chemistry and phytochemistry, for example extraction methods using polar or non-polar organic solvents (EP3682865).

However, we have observed an ever-increasing desire among consumers to turn to natural products containing as few synthetic ingredients as possible. To meet this new requirement, the extracts described in this application are 100% natural.

One approach is to use microorganisms to orchestrate plant biotransformation. Numerous processes involving selected or modified microorganisms are described in the literature and are used in an industrial environment. The most widespread are based on fermentation, known for ages as a food preservation process. The main advantages of fermentation are preservation, improvement of taste or even the nutritional quality of foods, due to the presence of a higher quantity of easily assimilated bioactive molecules.

In the field of cosmetics, microorganisms are widely used for their direct beneficial effects on the skin or to produce fermented or biotransformed plant extracts. We can cite for example the documents EP3744339A1 and CN110680774A which describe fermentation processes for plants, respectively Citrus auriantium and jasmine flowers, involving inoculation with yeasts of the species Saccharomyces cerevisiae or the document CN101243897A which describes the use of lactic acid bacteria to produce cosmetic or food flower extracts.

In the same field, document FR3103826 describes the preparation of a consortium of microorganisms composed of at least one lactic acid bacteria, one yeast and one acetic bacteria, to prepare plant extracts rich in active compounds, capable of being used in the pharmaceutical, cosmetic and food fields.

Biotechnological processes using microorganisms on synthetic nutrient media adapted to produce compounds of interest such as vitamins, citric acid, lactic acid or vanillin (US20060269632) are also described.

However, the processes cited above involve the exogenous supply of microorganisms or the supply of specific nutrients to stimulate the growth of microorganisms which will ensure the biotransformation of plant materials.

Fermentation can also rely on the endogenous microflora present on the surface or in the internal structures of plants. It is a community of bacterial and fungal, mutualistic or symbiotic microorganisms called phytobiota, hosted by almost all plants or parts of plants (fruits, leaves, flowers, stems, roots, seeds, mushrooms, algae). We then speak of spontaneous fermentation or autofermentation.

Thus, document US20060269632A1 describes the preparation of an effective medication against allergies obtained by mixing pine shoots with water and sugars and allowing spontaneous fermentation to develop for several months, in anaerobic conditions.

A problem that the invention proposes to solve is to provide a new process for preparing natural plant extracts, simple to implement, without adding any living microorganism, any enzyme, or any exogenous nutrient and nevertheless presenting remarkable biological effectiveness, as well as an absence of toxicity.

To solve the problem, the inventors have developed a process comprising a controlled autofermentation step carried out essentially from the phytobiota of plant materials and in particular flowers, fruits, leaves, and roots, without any exogenous input.

Under these conditions, a significant amount of organic acids and phenolic acids are generated. The plant extracts obtained then contain a wide range of phytomolecules giving them proven biological effectiveness.

The extracts thus obtained can be used in cosmetics for skin care and more particularly to combat the signs of skin aging, loss of firmness or tone, improve barrier function and hydration, lighten the skin or even improve the skin's innate immune defenses and in vivo well-being.

### SUMMARY OF THE INVENTION

The subject of the invention is an autofermentation process making it possible to obtain plant extracts enriched in organic acids and phenolic acids, comprising the following steps:
a) the plant matter is brought together with water,
b) if necessary, the pH is adjusted to a value between 4 and 9,
c) the mixture is kept under gentle stirring for a time of between 6 hours and 48 hours, at a temperature of between 2° and 60° C., at a pH of between 4 and 7, to allow the autofermentation process, in an enclosure allowing gas exchange with the atmosphere.
d) the mixture obtained in c) is purified to eliminate the residual solid plant material and harvest the liquid part,
e) at least one filtration is carried out, of the liquid part obtained in the previous step,
f) the pH of the filtrate is checked and readjusted, if necessary, to a value between 4 and 8 and preferably between 4 and 7.

The invention also relates to an autofermentation process coupled with a subsequent extraction allowing the enrichment of small molecular weight RNA, comprising the following steps:
a) the plant matter is brought together with water,
b) if necessary, the pH is adjusted to a value between 4 and 9,
c) the mixture is kept under gentle stirring for a time of between 6 hours and 48 hours, at a temperature of between 2° and 60° C., at a pH of between 4 and 7, to allow the autofermentation process, in an enclosure allowing gas exchange with the atmosphere.
d′) To the mixture obtained in step c) phytic acid is added at a concentration between 1 and 5 mM
e′) if necessary, the pH is adjusted to a value between 10 and 11,
f′) the mixture is kept stirring for 1 to 3 hours, at a temperature between 50° C. and 80° C.,
g′) the residual plant material is separated
h′) the liquid fraction obtained is purified by successive filtrations to clarify the extract,
i′) the pH of the mixture obtained is adjusted to a value between 6 and 8,
j′) Optionally, the extract is diluted with a physiologically acceptable solvent.

The invention also relates to diluted plant extracts, advantageously obtained from fresh jasmine flowers (Jasminum grandiflorum) by the autofermentation process coupled with a subsequent extraction allowing the enrichment of small RNAs above and comprising at least 300 mg/kg of phenolic compounds including at least 140 mg/kg of phenolic acids and at least 300 mg/kg of organic acids, including at least 50 mg/kg of shikimic acid and 10 mg/kg of quinic acid.

The invention also relates to a composition comprising an effective quantity of extract obtained by one of the preceding processes and a physiological medium.

The invention also relates to the cosmetic use of the preceding composition to combat the signs of skin aging, loss of firmness, improve the barrier function and hydration, lighten the skin, alleviate the reduction linked to age of the number or activity of mechanosensory touch receptors (Piezo1) and oxytocin receptors (OXTR), or even improve the skin's innate immune defenses and well-being in vivo.

The invention also relates to a composition comprising the plant extracts of the invention to improve the innate immune defenses of the skin.

### BRIEF DESCRIPTION OF DRAWINGS

FIG. 1 Protein profiles of jasmine extracts obtained by HPLC

FIG. 2 HPLC analysis of phenolic compounds present in jasmine extracts

FIG. 3 HPLC analysis of organic acids present in jasmine extracts

FIG. 4 Evaluation of an autofermented jasmine extract enriched with 2% small RNA on the expression of the Piezol receptor in reconstructed epidermis.

FIG. 5 Evaluation of an autofermented jasmine extract enriched with small RNA at 2% on the expression of the oxytocin receptor (OXTR) on human skin biopsies

FIG. 6 Evaluation of an autofermented jasmine extract enriched in small RNAs at 2% on the expression of oxytocin receptor (OXTR) messenger RNAs in cultured keratinocytes

FIG. 7 Evaluation of an autofermented jasmine extract enriched with small RNA at 2% on the expression of viperin in cultured keratinocytes.

FIG. 8 Evaluation of an autofermented jasmine extract enriched with small RNA at 2% on the expression level of E-cadherin, on human skin biopsies pretreated with a blocker of Piezol activity (Dooku1)

FIG. 9 Evaluation of an autofermented jasmine extract enriched with small RNAs at 2% on the expression level of messenger RNAs of the 11ß-HSD1 enzyme, in cultured keratinocytes.

FIG. 10 Evaluation of the effect of an autofermented jasmine extract enriched with small RNA at 2% on well-being by WHO questionnaire, by measurement of salivary oxytocin and measurement of emotional state.

FIG. 11 Evaluation of the effect of an autofermented jasmine extract enriched with small RNA at 2% of insensible water loss.

FIG. 12 Evaluation of the effect of an autofermented jasmine extract enriched with small RNA at 2% on ITA (Individual Topology Angle)

FIG. 13 Evaluation of the effect of an autofermented jasmine extract enriched with small RNA at 2% on the roughness of the skin on the forehead.

FIG. 14 Evaluation of the effect of an autofermented jasmine extract enriched with small RNA at 2% on the condition of the skin by the clinical expert.

FIG. 15 Diagram of the autofermentation process and the autofermentation process coupled with an extraction allowing the enrichment of small molecular weight RNA.

### DETAILED DESCRIPTION OF THE INVENTION

### Definitions

All terms used in this description have the most widely known meaning unless otherwise stated. For the purposes of the invention the following terms are defined as follows:

“Extract” means the result of all aqueous extraction processes from plant material.

By “phytobiota” we mean all the microorganisms present on the surface or inside plant material.

In the present description, “plant material” or “plant” means a living organism belonging to the plant kingdom and provided with a phytobiota, including, plants, moss, lichens and algae among others.

“Fresh plant material” means that the plant material used in the extraction process has not undergone any chemical or mechanical treatment likely to alter its phytobiota. For example, the plant material was harvested shortly before its engagement in the process or it was frozen quickly after its harvest or it was dried under conditions allowing good preservation of the phytobiota, for example at low temperature. Fresh plant material may include plant residues obtained after processing.

“Autofermentation” means a fermentation process resulting from the own phytobiota naturally present in or on the surface of the plant materials involved in the extraction process described in the present application.

By “proteins”, we mean large molecules composed of chains of amino acids, but also polypeptides and peptides of size less than 20 kDa (or 20 kg/mol).

By “small RNA” or “small molecular weight RNA” we mean a mixture of non-coding RNA (ribonucleic acids), of small molecular weight, with a length of a maximum of 150 nucleotides, such as all types of small non-messenger RNAs, single and/or double stranded, for example microRNAs, interfering RNAs, introns, small nuclear RNAs or even any RNA fragment.

By “organic acids” we mean derivatives of the catabolism of amino acids, fatty acids and sugars comprising an acid function. This group includes α-hydroxylated carboxylic acids (AHA) or polyhydroxylated acids such as glycolic, lactic, malic, citric, tartaric, shikimic, quinic acids, carboxylic acids derived from fruit sugars or any other parts of plants such as uronic acids, or even diacids such as succinic acid.

By “phenolic compounds” we mean all molecules having one or more aromatic rings bearing one or more hydroxyl groups, such as phenolic acids, flavonoids or their derivatives, tannins or any other polyphenols.

By “phenolic acids” we mean phenolic compounds derived from benzoic acid and cinnamic acid, molecules having a single aromatic ring.

By “flavonoids” we mean phenolic compounds all sharing the same basic structure formed by two aromatic rings linked by three carbons.

By “glycosylated flavonoids” we mean flavonoids linked to one or more sugars.

By “sugars” we mean all carbohydrates such as mono- and disaccharides as well as oligo- and polysaccharides contained in the extract.

By “jasmintides” we mean peptides from jasmine characterized by a composition rich in cysteine and non-classical disulfide bridges.

By “phytomolecules of interest”, we mean all the molecules present in the extracts of the invention and in particular, proteins, sugars, phenolic compounds, organic acids, small RNAs of a length of a maximum of 150 nucleotides.

When a range of values is described, the limits of this range must be understood as explicitly including all intermediate values of the range. For example, a range of values included between 1% and 10% must be understood to include 1%, 2%, 3%, 4%, 5%, 6%, 7%, 8%, 9%, and 10%, as well as all decimal values between 1% and 10%.

Numerical percentage values are percentages by weight, i.e. the weight of a compound relative to the total weight of the intended mixture, unless otherwise specified.

The compositions described herein may “comprise”, “consist of” or “consist substantially of” the essential compounds or optional ingredient.

The term “consist substantially of” means that the composition or component may include additional ingredients, but only if the additional ingredients do not alter the basic or novel characteristics of the composition or use described in this application.

A “physiologically acceptable medium” means a vehicle suitable for contact with the outer layers of the skin or mucous membranes, without causing toxicity, irritation, undue allergic and similar responses, or intolerance reactions, and proportionate to a reasonable benefit/risk ratio.

“Skin” means the skin of the face and body, including the scalp (including hair follicles and inter-follicular skin spaces) and appendages (hair, body hair and nails).

By “effective quantity” we mean the minimum quantity of extract according to the invention which is necessary to obtain at least one of the desired biological activities, without this quantity being toxic.

By “skin hydration”, we mean the water content and distribution of the upper layers of the epidermis.

By “signs of skin aging” we mean all changes in the external appearance of the skin due to aging such as, for example, wrinkles and fine lines, cracks, bags under the eyes, dark circles, wilting, loss of elasticity, firmness and/or tone of the skin, pigmentary disorders such as senile lentigo or solar lentigo, but also any internal changes in the skin which do not systematically result in a modified external appearance such as, for example, the reduction in the number or activity of mechanosensory touch receptors (Piezo1) and oxytocin receptors (OXTR), thinning of the skin, or any internal damage to the skin following environmental stresses such as pollution and solar radiation including UV.

By “well-being in vivo” we mean the emotional state of a person, the subjective feeling of well-being, the physiological production of oxytocin and the measurement of behavior or emotional expression.

It is understood that the invention relates to mammals and more particularly human beings.

### Autofermentation and Extraction Processes and Extracts Obtained

The subject of the invention is a process for obtaining plant extracts enriched in organic acids and phenolic acids comprising the following steps:
a) the plant matter is brought together with water,
b) if necessary, the pH is adjusted to a value between 4 and 9,
c) the mixture is kept under gentle stirring for a time of between 6 hours and 48 hours, at a temperature of between 2° and 60° C., at a pH of between 4 and 7,
d) the mixture obtained in c) is purified to eliminate the residual solid plant material and harvest the liquid part.
e) We carry out at least one filtration of the liquid part obtained in the previous step.
f) The pH of the filtrate is checked and readjusted, if necessary, to a value between 4 and 8.

It is the fresh plant material placed in the presence of water which provides the nutrients necessary for the growth of the phytobiota. The nutrient resource is sufficient to allow the development and survival of the phytobiota for several days.

The process of the invention is implemented without the addition of exogenous nutrients and does not use detergents or potentially toxic solvents in cosmetics. It is therefore simple to implement and has a reduced impact on the environment.

In step c), moderate agitation keeps the phytobiota microorganisms suspended in the mixture.

The autofermentation process of step c) is carried out in an open environment to allow gas exchange with the atmosphere, without any particular constraints to ensure sterility, but can alternatively be implemented in a fermenter in order to control gas exchanges.

Autofermentation takes place when the microorganisms of the phytobiota, through their enzymes, metabolize nutrients of plant origin, such as sugars, proteins, polyphenols to produce an extract rich in various families simpler and more easily assimilated compounds such as organic acids, peptides, and phenolic acids.

A study of the kinetics of microbial growth coupled with tests made it possible to observe a significant modification of the medium during autofermentation, characterized by a drop in the flavonoid content, a drop in the concentration of sugars, in particular glucose and the increase in fermentation markers such as phenolic and organic acids attesting to the fermentative activity of microorganisms.

In a particular embodiment, the plant materials used are plants of the Oleaceae, Poaceae or Fabaceae family.

In another particular embodiment, the plant materials used are plants of the Oleaceae family such as jasmine of the species Jasminum grandiflorum.

In a particular embodiment, the plant parts used are chosen from flowers, fruits, leaves, and roots.

In a preferred embodiment, the plant parts used are the flowers.

In another embodiment, the plant materials used are algae.

In another particular embodiment, the plant material is a plant residue obtained after processing such as cakes or spent grains.

In step a) we use distilled, demineralized water or water rich in mineral salts and/or trace elements. The water preferably used is distilled water.

The plant material used in step a) can be whole or mechanically crushed using a blade grinder for example to reduce the particle size from 0.5 mm to a few centimeters without affecting the viability of the plants, microorganisms. Grinding can be carried out dry or in water.

When the plant material is composed of flowers, it is preferably used whole, that is to say not crushed. Indeed, comparative extractions carried out with or without grinding have shown that the passive diffusion of the compounds contained in the flowers is high enough to make grinding unnecessary.

On the other hand, when the starting plant material is too thick or composed of large fragments, for example, a whole alga or a root, it is preferably crushed before step a).

In step a) the plant material/water ratio is preferably between 3 to 30% w/w, more preferably between 3 and 15%, even more preferably it is 3%, 5% or 15%.

At the start of step b) the pH is controlled and adjusted to a value between 4 and 9, preferably between 6 and 8 and even more preferably at pH 7, by the addition of hydrochloric acid (HCl), citric acid or sodium hydroxide (NaOH). The pH is then checked regularly throughout step b) and possibly readjusted.

When citric acid is used in this step, this amount of citric acid is removed in the final organic acid concentration calculations of the extract.

In step c), the agitation of the mixture is advantageously moderate agitation of the stirring type. The temperature is adjusted and maintained between 2° and 60° C., more preferably between 2° and 40° C., and even more preferably between 3° and 40° C. These temperature ranges are ideal for ensuring the optimal development of micro-organisms, in fact below 20° C. microbial growth is slowed and above 60° C. the viability of micro-organisms decreases.

Step c) must be carried out at an optimal pH and temperature to enrich the aqueous extraction solution with phytomolecules and create the conditions for growth of the phytobiota present in the mixture. During this stage the pH must not fall below 5.

Autofermentation step c) takes place for a period of between 1 and 48 hours, preferably between 6 and 30 hours, and more preferably between 15 and 30 hours. A study of microbial growth kinetics demonstrated that growth is strong, under process conditions, for a period of at least 30 hours, then reaches a plateau.

Optionally, at the end of step c) the residual solid material can be crushed to release the cellular content of the microorganisms and thus increase the extraction yield of the phytomolecules.

To carry out step d) any method known to those skilled in the art may be used. The mixture obtained in c) can, for example be filtered on filters with porosity greater than 30 μm. Preferably the mixture obtained in c) is centrifuged at low speed, for example for at least 10 min at 4000 g.

To carry out step e), successive filtrations are preferably carried out by lowering the filtration threshold from 30 to 1 μm in order to clarify the mixture. Preferably, at least 4 filtrations are carried out with filters with porosities ranging from 30 μm, 4 μm, 2 and 1 μm.

In step f) the pH of the filtrate is checked and readjusted, if necessary, to a value between 4 and 8, and preferably to a value between 4 and 7. The pH can be adjusted by adding a solution of sodium hydroxide (NaOH) or hydrochloric acid (HCl) or any other equivalent acid, compatible with cosmetic use, such as citric acid. This step is essential to avoid the precipitation of phytomolecules of interest such as sugars, phenolic compounds, organic acids, proteins and thus obtain a stable extract. Preferably, the pH is adjusted to a value between 4 and 7.

In step g) the filtrate can optionally be sterilized by any method known to those skilled in the art, for example by steaming or preferably by sterilizing filtration on a 0.2 to 0.45 μm filter.

At the end of step f) or g), a concentrated autofermented extract is obtained.

This concentrated autofermented extract is characterized by a dry weight of 4 to 30 g/kg, 0.5 to 6 g/kg of sugars, 300 to 900 mg/kg of organic acids including 50-500 mg/Kg of shikimic acid and 10 to 100 mg/Kg of quinic acid, 300 to 2000 mg/Kg of phenolic compounds including 100 to 200 mg/Kg of phenolic acids and 1 to 10 g/Kg proteins. However, the extracts obtained may exhibit significant variability depending on factors such as location or year of harvest, season, climatic conditions, biotic stress, etc.

The autofermented extract does not contain ethanol since the process of the invention is not an alcoholic type of fermentation.

h) The concentrated autofermented extract thus obtained can be diluted in a physiologically acceptable solvent for cosmetic use.

The autofermentation process allows the enrichment of the extract in phenolic acids resulting from the transformation of polyphenols and in organic acids such as quinic, succinic and shikimic and lactic acids.

Thus, analysis by high performance liquid chromatography showed that unexpectedly the concentrated autofermented extracts obtained by the process described above contain at least 300 mg/kg of phenolic compounds including at least 100 mg/kg of phenolic acids and at least 300 mg/kg of organic acids including at least 50 mg/kg of shikimic acid and 10 mg/kg of quinic acid.

Indeed, generally speaking, the analysis of organic acids contained in flowers and in particular in lavender flowers, do not contain shikimic and quinic acids.

In step h) among the physiologically acceptable solvents, mention may be made of water, glycerol, ethanol, propanediol as well as its natural version from corn, butylene glycol, dipropylene glycol, diglycols ethoxylated or propoxylated, cyclic polyols or any mixture of these solvents. One of the advantages of such a dilution, in addition to obtaining exactly the desired concentrations of phytomolecules, is to improve the stability and conservation of the autofermented plant extract.

Preferably, the concentrated autofermented extract is diluted with butylene glycol, propanediol or even glycerin and even more preferentially diluted by adding 30% propanediol to ensure its stability and conservation over time by preventing contamination. The concentrated extract can also be diluted by adding 30% glycerin combined with all types of water-soluble preservatives such as sodium benzoate or potassium sorbate at the final concentration of 0.5% or even phenoxyethanol at the final concentration of 1.5%.

This so-called diluted extract comprises by weight of the total weight of the extract from 2 to 20 g/kg of dry extract, 0.2 to 4 g/kg of sugars, 100 to 700 mg/kg of organic acids including 50 to 350 mg/Kg of shikimic acid and 10 to 70 mg/Kg of quinic acid, 100 to 1300 mg/kg of phenolic compounds including 70 to 140 mg/Kg of phenolic acids and 0.1 to 5 g/Kg of protein.

The extract of the invention thus comprises a wide range of phytomolecules that can have beneficial effects on the skin, without presenting a risk of skin irritation or other damage to health.

In this particular embodiment, the plant material is advantageously a plant from the Oleaceae, Poaceae or even Fabaceae family.

In this particular embodiment, the plant material is even more advantageously fresh jasmine flower, preferably of the species Jasminum grandiflorum.

In addition, after step c), additional extraction steps can be carried out according to any method known to those skilled in the art.

Among these additional extraction steps, we can cite an extraction process allowing the enrichment of small molecular weight RNA described in patents FR 1670672 and U.S. Pat. No. 11,021,505.

To carry out this process, the following steps are carried out:
Step d′) To the mixture obtained in step c) phytic acid is added at a concentration between 1 and 5 mM.
Step e′) the pH is adjusted, if necessary, to a value between 10 and 11.
Step f′) the mixture is kept stirring for 1 to 3 hours, preferably for 2 hours, at a temperature between 50° C. and 80° C., preferably at 80° C.
Step g′) the residual plant material is removed.
Step h′) the liquid fraction obtained is purified by successive filtrations to clarify the extract.
Step i′) the pH of the mixture obtained is adjusted to a value between 6 and 8.

One of the advantages of coupling the autofermentation process with a complementary extraction process allowing the enrichment of small RNA is to increase the extraction yield by at least a factor of 2 in comparison with an extract obtained by autofermentation. The concentration of proteins, sugars, organic acids, and total phenolic compounds is also increased by approximately a factor of 2 to 3.

Thus, the concentrated extracts enriched in small RNAs obtained by the processes described above have a dry weight at least twice greater than the dry weight of the extracts obtained by simple autofermentation and contain at least 500 mg/kg of phenolic compounds of which at least less 600 mg/kg of organic acids.

Such concentrated extracts are characterized by a dry weight of 4 to 60 g/kg and include 0.5 to 10 g/kg of sugars, 600 to 1200 g/kg of organic acids including 50 to 700 mg/kg. of shikimic acid and 20 to 200 mg/Kg of quinic acid, 500 to 3000 mg/Kg of phenolic compounds including 200 to 300 mg/Kg of phenolic acids, 2 to 20 g/Kg proteins, as well as 10 to 100 mg/Kg of small molecular weight RNA.

In this particular embodiment, the plant material is advantageously a plant from the Oleaceae, Poaceae or even Fabaceae family.

In this particular embodiment, the plant material is even more advantageously fresh jasmine flower, preferably of the species Jasminum grandiflorum.

When the plant material is fresh jasmine flower (Jasminum grandiflorum), the extract obtained has a dry weight at least 2 times greater than the dry weight of extracts obtained by simple autofermentation and comprises at least 500 mg/kg of compounds phenolics, at least 600 mg/kg of organic acids and at least one jasmintide.

Jasmintides are part of the cyclotide family which have an activity close to oxytocin in relation to well-being and anti-aging effects (Adriano Mollica et al. 2015, Cyclotides: a natural combinatorial peptide library or a bioactive sequence player? Journal of Enzyme Inhibition and Medicinal Chemistry, 30:4, 575-580).

Step j′) Optionally, the extract is diluted with a physiologically acceptable solvent.

To carry out this dilution step, the physiologically acceptable solvent is chosen from water, glycerol, ethanol, propanediol as well as its natural version from corn, butylene glycol, dipropylene glycol, ethoxylated diglycols or propoxylated, cyclic polyols or any mixture of these solvents. One of the advantages of such a dilution, in addition to obtaining exactly the desired concentrations of phytomolecules, is to improve the stability and conservation of the autofermented plant extract.

Preferably, the autofermented extract enriched in concentrated small RNA is diluted with butylene glycol, propanediol or even glycerin and even more preferentially diluted by adding 30% propanediol to ensure its stability and conservation over time by preventing contamination. The concentrated extract can also be diluted by adding 30% glycerin combined with all types of water-soluble preservatives such as sodium benzoate or potassium sorbate at the final concentration of 0.5% or even phenoxyethanol at the final concentration of 1.5%.

After dilution in a physiologically acceptable solvent, such extracts are characterized by a dry weight of 2 to 40 g/kg and include 0.2 to 5 g/kg of sugars; 300 to 900 mg/kg of organic acids including 50 to 500 mg/kg of shikimic acid and 10 to 200 mg/kg of quinic acid; 300 to 2000 mg/kg of phenolic compounds including 140 to 210 mg/Kg of phenolic acids, 2 to 10 g/kg of proteins as well as at least 10 to 60 mg/Kg of small molecular weight RNA.

When the plant material is fresh jasmine flower (Jasminum grandiflorum), the extract also comprises at least one jasmintide.

### Cosmetic Composition

Another object of the invention is a cosmetic composition comprising an effective quantity of at least one plant extract obtained according to the process described in the present application.

Advantageously, the plant extracts of the invention are used in diluted form and are added to a physiologically acceptable medium at a concentration of 0.05 to 5% by weight relative to the total weight of the composition, preferably at a concentration of 0.1 to 2.5% by weight relative to the total weight of the composition.

The composition usable according to the invention is formulated to be applied by any appropriate route, in particular oral, or external topical, and the formulation of the compositions will be adapted by those skilled in the art.

Preferably, the compositions according to the invention are in a form suitable for topical application. These compositions must therefore contain a physiologically acceptable environment, that is compatible with the skin and appendages, without risk of discomfort during their application and cover all suitable cosmetic forms.

The compositions for implementing the invention may in particular be in the form of an aqueous, hydroalcoholic or oily solution, an oil-in-water, water-in-oil emulsion or multiple emulsions; they can also be in the form of suspensions, or even powders, suitable for application to the skin, mucous membranes, lips and/or hair.

These compositions can be more or less fluid and also have the appearance of a cream, a lotion, a milk, a serum, an ointment, a gel, a paste or a foam. They can also be in solid form, like a stick or applied to the skin in aerosol form.

As a physiologically acceptable medium commonly used in the envisaged field of application, for example, of adjuvants necessary for the formulation, such as solvents, thickeners, diluents, antioxidants, dyes, sunscreens, self-tanning agents, pigments, fillers, preservatives, fragrances, odor absorbers, essential oils, vitamins, essential fatty acids, surfactants, film-forming polymers, etc.

In all cases, those skilled in the art will ensure that these adjuvants as well as their proportions are chosen in such a way as not to harm the desired advantageous properties of the composition according to the invention. These adjuvants can, for example, correspond to 0.01 to 20% of the total weight of the composition. When the composition according to the invention is an emulsion, the fatty phase can represent 5 to 80% by weight and preferably 5 to 50% by weight relative to the total weight of the composition. The emulsifiers and coemulsifiers used in the composition are chosen from those conventionally used in the field considered. For example, they can be used in a proportion ranging from 0.3 to 30% by weight relative to the total weight of the composition.

According to another advantageous embodiment of the invention, the plant extracts of the invention can be encapsulated or included in a cosmetic vector such as liposomes or another nano capsule or microcapsule used in the field of cosmetics or adsorbed on powdery organic polymers, mineral supports such as talcs and bentonites.

Advantageously, the composition according to the invention may comprise, in addition to the plant extract according to the invention, at least one other active agent presenting cosmetic effects similar and/or complementary to those of the invention.

For example, the additional active agent(s) may be chosen from: anti-aging, firming, lightening, moisturizing, draining, microcirculation promoting, exfoliating, desquamating, stimulating the extracellular matrix agents, activating energy metabolism, antibacterial, antifungal, soothing, anti-radical, anti-UV, anti-acne, anti-inflammatory, anesthetic, providing a feeling of heat, providing a feeling of freshness, slimming,

Such additional active agents can be chosen from the groups comprising:
vitamins (vitamin A and its derivatives; vitamins B3, B5, B6 and B12; vitamin C; vitamins E, F, H, K, PP or even coenzyme Q10);
metalloproteinase inhibitors, or TIMP activators;
DHEA, its precursors and derivatives;
amino acids, natural or synthetic peptides,
Artemia salina extract, marketed under the name GP4G™ (FR2817748, ASHLAND®);
plant peptide extracts, yeast extracts, polyphenol extracts;
dehydroacetic acid (DHA);
phystosterols of synthetic or natural origin;
salicylic acid and its derivatives, alpha- and beta-hydroxy acids, silanols;
amino sugars and polysaccharides;
lipids such as ceramides or phospholipids;
cyclic AMP and its derivatives, methyl xanthines.

### Cosmetic Uses

The invention also relates to the cosmetic use of a composition comprising the plant extracts of the invention for skin care and more particularly for combating the signs of skin aging, loss of firmness or tone, improving barrier function and hydration, lighten the skin, alleviate the age-related decrease in the number or activity of mechanosensory touch receptors (Piezo 1) and oxytocin receptors (OXTR), or improve the skin's innate immune defenses and in vivo well-being.

Preferably, the cosmetic uses according to the present invention relate to cosmetic treatment methods by topical applications on healthy skin.

In a particular embodiment, the invention relates to the cosmetic use of autofermented extracts of fresh jasmine flowers (Jasminum grandiflorum).

Advantageously, the subject of the invention is the cosmetic use of a composition comprising an extract of fresh jasmine flowers (Jasminum grandiflorum) obtained by a autofermentation process coupled with an extraction process allowing the enrichment of small RNA molecular weights (process described in patents FR 1670672 and U.S. Pat. No. 11,021,505).

The plant extracts of the invention were tested on biological markers associated with aging and hydration, such as the expression of collagen and hyaluronic acid, and demonstrated greater effectiveness than conventional control plant extracts.

The plant extracts of the invention were tested on biological markers associated with the firmness or tone of the skin, such as e-cadherin and demonstrated greater effectiveness than conventional control plant extracts.

The plant extracts of the invention were tested on tyrosinase, an enzyme associated with the synthesis of melanin, and therefore with skin pigmentation, and showed greater tyrosinase inhibitory activity than conventional control plant extracts.

A cosmetic composition comprising the plant extracts of the invention made it possible to alleviate the age-related decrease in the number or activity of mechanosensory touch receptors (Piezo 1).

A cosmetic composition comprising the plant extracts of the invention made it possible to stimulate the expression of the oxytocin receptor, a molecule involved in the prevention of skin aging.

In this particular embodiment, jasmine flower extracts have demonstrated their ability to stimulate the expression of viperin, a molecule associated with the skin's innate immune defenses.

In this particular embodiment, a composition comprising 2% extracts of the invention, applied topically by healthy volunteers, produced a positive effect on well-being in vivo. In this test, emotional state was assessed through three distinct components: subjective feeling of well-being, salivary oxytocin production and measurement of behavior or emotional state (Don Hockenbury and Sandra E. Hockenbury, Discovering psychology, 5th edition, Page 344, chapter 8, Worth Publishers).

In a particular embodiment the invention also relates to a composition comprising the plant extracts of the invention to improve the immune defenses of the skin.

### EXAMPLES

By way of illustration, examples of embodiments of the method according to the invention are described below.

### Example 1: Preparation of an Extract of Fresh Autofermented Jasmine Flowers (Jasminum grandiflorum)

The species Jasminum grandiflorum belongs to the genus Jasminum and is widely used in industry as a fragrance or aroma.

In a first step a), 200 g of whole fresh jasmine flowers are mixed with 1800 g of distilled water, i.e. 10% of raw material used in the process and 90% water for a total weight of 2 kg.
b) The pH is adjusted to 7.
c) The mixture is maintained at 30° C., in an open beaker to maintain an aerobic atmosphere, favorable to the development of microorganisms for 24 hours. During this stage, the pH is checked regularly, allowing us to note an acidification of the environment linked to the development of microorganisms, without it falling below 5.
d) The mixture obtained is centrifuged for 10 min at 4000 g, to sediment the residual plant material in the pellet and collect the supernatant.
e) The mixture obtained is then subjected to successive filtrations by lowering the filtration threshold from 30 to 0.2 pm.
f) The pH of the filtrate obtained is adjusted to 6.3 using 10% concentrated citric acid.
g) The filtrate is sterilized by filtration.

The undiluted extract has a dry weight of 11.7 g/kg and contains a concentration of 5.2 g/kg total sugars, 3.7 g/kg protein, 411.1 mg/kg total organic acids, 630 mg/kg total phenolic compounds.
h) The extract is diluted with plant-derived glycerin to obtain a final concentration of 30% glycerin and 70% autofermented jasmine flower extract

The diluted extract has a dry weight of 8.1 g/kg and has a concentration of 3.6 g/kg of total sugars, 2.6 g/kg of proteins, 287.7 mg/kg of organic acids. total, 441 mg/kg of total phenolic compounds.

### Example 2: Preparation of an Extract of Fresh Jasmine Flowers (Jasminum Grandiflorum) by Autofermentation Coupled with a Second Extraction Process Allowing Enrichment in Small RNAs

Once the autofermentation has been carried out according to steps a) to c) of example 1.

2 g/L or 3 mM of phytic acid are added to the mixture.

The pH is adjusted to pH 11 in order to allow enrichment of the extract in small molecular weight RNA as well as in various phytomolecules.

The mixture is heated for 2 hours at 80° C. under stirring.

The mixture is then filtered using filters with a porosity of 30 μm, to separate the solid material from the filtrate.

Sequential filtrations on filters of decreasing porosity are then carried out to clarify the plant extract until filtration at 1 μm porosity.

The pH is adjusted to 6.3 with a citric acid solution.

At this stage, the extract has a dry weight of 17.5 g/kg and has a concentration of 6.4 g/kg of total sugars, 6.6 g/kg of proteins, and 802.9 mg/kg of total organic acids, 1301 mg/kg of total phenolic compounds and 45 mg/kg of small molecular weight RNA and is free from DNA.

The absence of DNA was demonstrated by a DNAse test, an enzyme which specifically degrades DNA and not RNA. The electrophoretic profile after DNase action is not modified, which demonstrates that the nucleic acid present in the extract is not sensitive to DNase and is therefore not DNA.

The extract is then diluted with plant-derived propanediol to obtain a final concentration of 30% propanediol and 70% autofermented and extracted jasmine flower extract.

The diluted extract has a dry weight of 11.7 g/kg and has a concentration of 4.1 g/kg of total sugars, 4.6 g/kg of proteins, 562.5 mg/kg of total organic acids, 874 mg/kg of total phenolic compounds and 31 mg/kg of small molecular weight RNA.

### Example 3: Preparation of Jasmine Extracts as Comparative Elements

### 1/ Extraction of Fresh Jasmine Flowers Using a Conventional Process:

For comparative purposes, conventional extracts were produced using the same quantity of whole, fresh jasmine flowers as in Examples 1 or 2, i.e. 10% of plant raw material in distilled water.

The mixture is then heated for 1 hour at 25° C., then filtered by a first filtration on large porosity filters of 30 μm in order to remove the solid residual plant material from the liquid part. Then sequential filtrations on filters of decreasing porosity are then carried out in order to clarify the plant extract until sterilizing filtration at 0.2 m porosity.

The liquid part obtained in the previous step constitutes a conventional control extract.

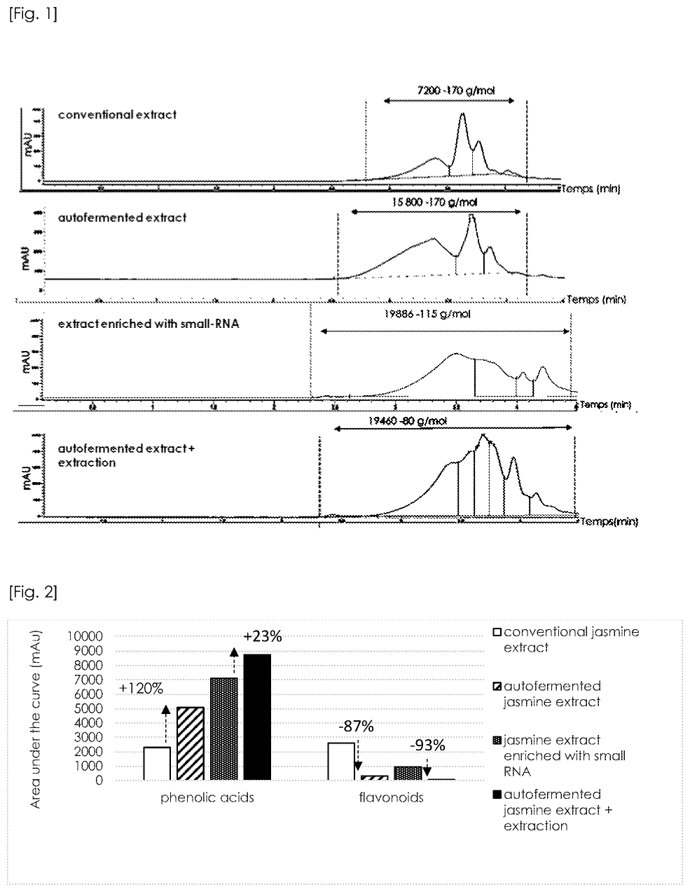

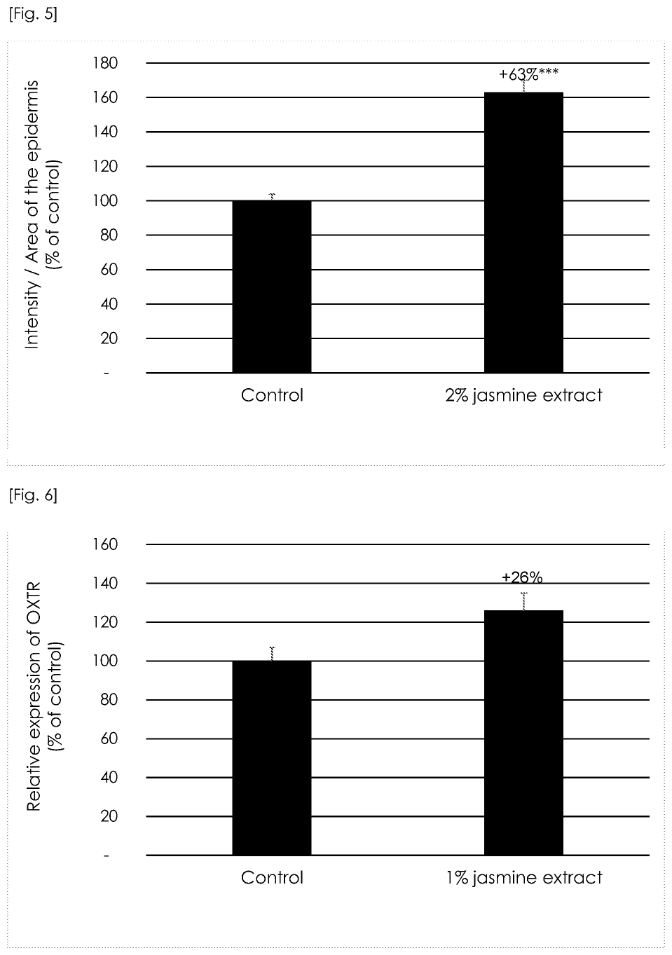

Comparative analytical data could thus be generated, as illustrated in [FIG. 1], [FIG. 2], [FIG. 5] and examples.

### 2/ Extraction of Fresh Jasmine Flowers by a Process Allowing the Enrichment of Small RNAS:

This process is described in detail in patents FR 1670672 and U.S. Pat. No. 11,021,505.

To obtain the fresh flower extract enriched in small RNA, the same quantity of whole, fresh jasmine flowers is used as in examples 1 and 2, i.e. 10% of plant raw material in distilled water.

2 g/L or 3 mM of phytic acid are added to the mixture.

The pH is adjusted to pH 11 in order to allow enrichment of the extract in small molecular weight RNA as well as in various phytomolecules.

The mixture is heated for 2 hours at 80° C. with stirring.

The mixture is then filtered using filters with a porosity of 30 μm, to separate the solid material from the filtrate.

Sequential filtrations on filters of decreasing porosity are then carried out in order to clarify the plant extract until filtration at 1 μm porosity.

The pH is adjusted to 6.3 with a citric acid solution.

The liquid part obtained in the previous step constitutes an extract enriched in small control RNAs.

Comparative analytical data could thus be generated, as illustrated in [FIG. 1], [FIG. 2], [FIG. 5].

### Example 4: Analysis of Jasmine Flower Extracts from Examples 1 and 2 Versus a Conventional Extract and an Extract Enriched in Small RNA from Example 3

The analyzes were carried out on the extracts of Examples 1 and 2 before dilution, on a conventional extract and on an extract enriched in small RNAs from Example 3.

### Methodologies

The total content of phenolic compounds in the extracts was measured spectrophotometrically at 760 nm after reduction of the Folin-Ciocalteu reagent by phenols. Quantification is carried out using a gallic acid standard curve, the results are expressed in gallic acid equivalent.

The total protein content was measured spectrophotometrically at 550 nm after calorimetric reaction of Biuret combined with that of the Folin-Ciocalteu reagent. Quantification was carried out using a BSA (Bovine Serum Albumin) standard curve.

The detailed composition of phenolic compounds was carried out by liquid chromatography coupled with a UV detector fixed at 254 nm. The samples were separated on an UPTISPHERE CS EVOLUTION CI 8-AQ column by an Agilent 1200 HPLC system (Agilent Technologies). The mobile phases consisted of a solution of 0.1% formic acid and methanol.

The molecular weight distribution profile of the proteins was carried out by size exclusion chromatography coupled with a UV detector fixed at 254 nm. The samples were separated on a YMC Pack Diol 60 column (DL06503-2543WT) by an Agilent 1200 HPLC system (Agilent Technologies). The mobile phase consisted of an aqueous solution of 0.05% NaN₃, 0.2 M NaCl, 0.1 M phosphate buffer at pH 7.

The characterization and quantification of organic acids was carried out by high-performance liquid chromatography, coupled with detection by mass spectrometer (Acquity Qda, Waters) equipped with an electrospray ion source in negative mode. The samples were separated on an EC 150/4.6 Nucleoshell RP 18plus-5|jm column (Macherey Nagel: 763236.46) by an Agilent 1260 HPLC system (Agilent Technologies). The flow rate was 0.3 ml/min. The mobile phases consisted of a solution of 0.01% formic acid and acetonitrile.

The characterization and quantification of sugars were carried out by high-performance liquid chromatography, coupled with detection by mass spectrometer (Acquity Qda, Waters) equipped with an electrospray ion source in negative mode. The samples were separated on a Luna Omega Sugar 100 A-3 μm column (Phenomenex: 00F-4775-E0) by an Agilent 1260 HPLC system (Agilent Technologies). The flow rate was 0.8 ml/min. The mobile phases consisted of an aqueous solution of 10 mMol ammonium acetate and 10 mMol ammonium acetate acetonitrile.

The identification of jasmintides was carried out by ultra-high performance liquid chromatography coupled with detection by high-resolution mass spectrometer. The samples were denatured and then separated on an Aurora column (15 cm×75m×1.6 μm×120A). The flow rate was 0.3 ml/min. The mobile phases consisted of an aqueous solution of 0.1% formic acid and acetonitrile with 0.1% formic acid. The identification of jasmintides was carried out by comparison with the Swissprot database.

The overall screening of the extracts was carried out by Gas Chromatography (GC) coupled with a Mass Spectrometer (MS). The samples were derivatized with (N,O-bis(trimethylsilyl) trifluoroacetamide (BSTFA) then separated on an Optima 5HT column (Macherey Nagel 726104.15) by a gradient at 3° C./min from 50° C. to 300° C.

### Results

The protein profile of the extracts is shown in [FIG. 1]. The molecular weights of the conventional extract are distributed between 7200 and 170 g/mol. In comparison, the autofermentation process of Example 1 provides a broadening of the molecular weight distribution of the proteins in the extract with masses ranging from 15,800 to 170 g/mol. Comparisons of protein profiles highlight the increase in the quantity of proteins during the fermentation process compared to a conventional extract for masses greater than 1000 g/mol.

The process of Example 2 (autofermentation coupled with extraction) increases the complexity of the extract with the appearance of proteins with a molecular weight around 19,000 g/mol and an increase in the signal for small molecules having a molecular weight around 1000 g/mol compared to the conventional extract. The quantification of total proteins shows an increase of 50% between the extract of example 2 (autofermented coupled with an extraction) and the extract enriched in small RNA.

The autofermentation process therefore modifies the protein profile of the extract and the coupling with an extraction step makes it possible to increase the total protein concentration.

Analysis of the total quantity of phenolic compounds indicates that the extract of Example 2 (autofermentation coupled with extraction) contains more phenolic compounds than a conventional extract (Example 3). [FIG. 2] shows a targeted consumption of complex phenolic compounds (flavonoids or glycosylated flavonoids) and the appearance of simple phenolic compounds such as phenolic acids during fermentation. [FIG. 2] indicates an increase of 120% in the presence of phenolic acids between the conventional extract and the autofermented extract and an increase of 23% between the extract enriched in small RNA and the extract of example 2 (autofermented coupled to an extraction). [FIG. 2] also indicates a reduction of 87% in the presence of flavonoids between the conventional extract and the autofermented extract and a reduction of 93% between the extract enriched in small RNAs and the extract of example 2 (autofermented coupled to an extraction).

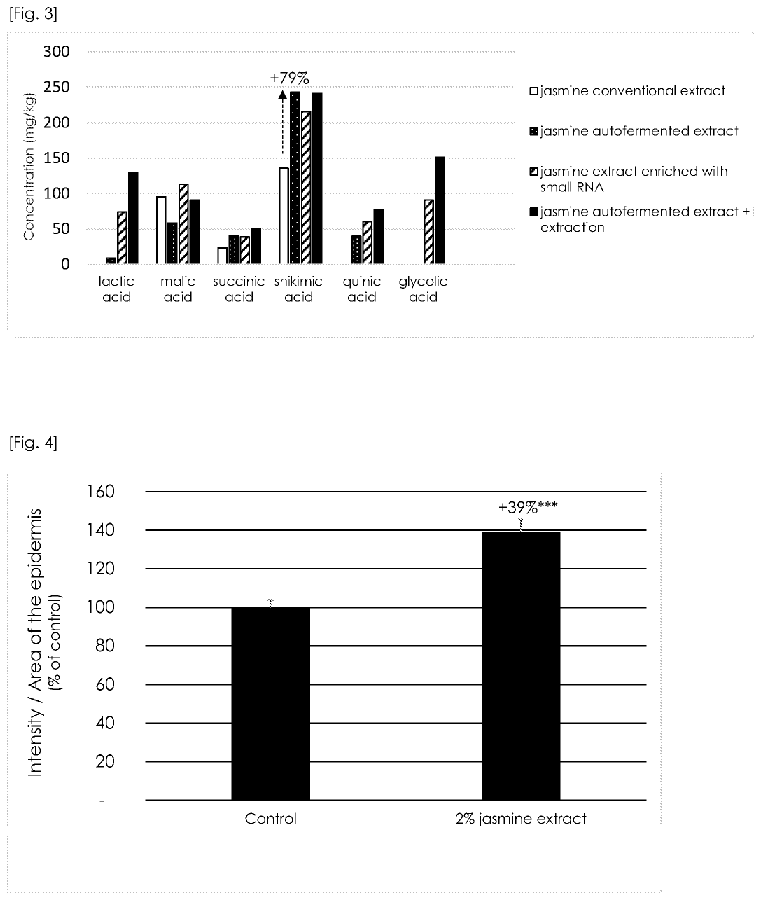

The analysis of the organic acids contained in the extracts, as illustrated by [FIG. 3], shows the increase in the concentration of lactic, succinic, shikimic, quinic and glycolic acids during the fermentation process.

[Table 1] details the presence of lactic and quinic acids in the autofermented extract versus their absence in the conventional extract and the increase of 72% and 79% in succinic and shikimic acids in the autofermented extract versus the conventional extract respectively. It also details the increase of 75%, 31%, 12%, 66% and 27% in the concentration of lactic, succinic, shikimic, glycolic and quinic acids in the autofermented extract coupled with an extraction versus an extract enriched in small RNA respectively.

The concentration of malic acid undergoes a reduction during the fermentation process with a reduction of 38% in the autofermented extract versus the conventional extract and a reduction of 19% in the autofermented extract coupled with an extraction versus the extract enriched with small RNAs.

The overall concentration of organic acids is higher in the autofermented extract coupled with an extraction than in conventional extracts enriched with small RNA which do not contain a fermentation step.

The analysis of the sugars shows a reduction of 54% in glucose in the extract of Example 2 (autofermented coupled with an extraction) versus the extract enriched in small RNA due to the sugar consumption of the microorganisms in the autofermented extract followed by extraction.

The overall concentration of phenolic acids is 57% higher in the autofermented extract coupled with an extraction than in the autofermented extract alone.

The analysis of jasmintides highlights the presence of at least one jasmintide in the extract of Example 2 (autofermented coupled with an extraction) as well as the absence of jasmintides in the conventional extract.

Analysis of the extracts by GCMS indicates that the autofermentation step makes it possible to increase the diversity of molecules present in the extract. In fact, 109 molecules are detected on the profile of the autofermented extract compared to 95 in the conventional extract. 165 molecules are detected in the extract of example 2 (autofermented coupled with an extraction) against 132 in the extract enriched in small RNAs. Coupling the autofermentation process with an extraction makes it possible to obtain an extract with a more diverse composition than conventional extractions and an enrichment in small RNAs.

TABLE 1
Analysis of compounds in jasmine flower extracts

| | Con- | Small RNA | Auto- | Autofermented |
|---|---|---|---|---|
| | ventional | enriched | fermented | extract + |
| | extract | extract | extract | extraction |
| Phenolic | 783 | 1278.9 | 630 | 1301 |
| compounds | | | | |
| (mg/kg) | | | | |
| Proteins | 4.7 | 4.4 | 3.7 | 6.6 |
| (g/kg) | | | | |
| Glucose | 2272 | 1914 | 2306 | 873 |
| (mg/Kg) | | | | |
| Shikimic acid | 135.7 | 215.8 | 243.2 | 241.4 |
| (mg/Kg) | | | | |
| Quinic acid | <DL | 60.2 | 39.8 | 76.6 |
| (mg/Kg) | | | | |
| Glycolic acid | Not | 90.8 | Not | 151.1 |
| (mg/Kg) | detected | | detected | |
| Lactic acid | Not | 74.0 | 8.7 | 129.5 |
| (mg/Kg) | detected | | | |
| Malic acid | 95.5 | 113.0 | 58.4 | 91.1 |
| (mg/Kg) | | | | |
| Succinic acid | 23.4 | 38.7 | 40.2 | 50.7 |
| (mg/Kg) | | | | |
| Phenolic | 65.5 | 201.1 | 143.6 | 247.1 |
| acids (mg/Kg | | | | |
| gallic acid | | | | |
| equivalent) | | | | |
| Small RNA | Not | 67.6 | Not | 34.7 |
| (mg/Kg) | detected | | detected | |

TABLE 2
Analysis of phenolic compounds in jasmine flower
extracts (area under the curve)

| | Con- | Small RNA | Auto- | Autofermented |
|---|---|---|---|---|
| | ventional | enriched | fermented | extract + |
| | extract | extract | extract | extraction |
| Phenolic | 2304 | 7115 | 5078 | 8749 |
| acids (mAU) | | | | |
| Flavonoids | 2606 | 947 | 329 | 71 |
| and | | | | |
| glycosylated | | | | |
| flavonoids | | | | |
| (mAU) | | | | |

### Example 5: Highlighting the Role of the Phytobiota in the Autofermentation of Jasmine Flowers (Jasminum grandiflorum)

Fresh jasmine flowers were sterilized by autoclaving for 10 minutes at 121° C. to kill microorganisms. This plant material deprived of its living phytobiota was then used in the process described in Example 1 to constitute a control extract.

The physicochemical analysis shows that the levels of phenolic compounds and proteins decrease sharply during the autofermentation process, while they are stable in the control condition.

These results demonstrate that the microorganisms of the phytobiota are directly responsible for the metabolic consumption of phenolic compounds and proteins during the autofermentation process described in the present application and in particular in Example 1.

TABLE 3
Comparison of the consumption of phenolic compounds and proteins in
autofermentative or control situation (killed phytobiota)

| | Autofermented jasmine | | Sterilized jasmine flowers | |
|---|---|---|---|---|
| | flowers extract | | extract | |
| | Start of | End of | Start of | End of |
| | fermentation | fermentation | fermentation | fermentation |
| Phenolic | 912 | 486 | 1046 | 980 |
| compounds | | | | |
| (mg/kg) | | | | |
| Proteins (g/kg) | 4.1 | 2.9 | 5 | 5.1 |

### Example 6: In Vitro Evaluation of the Hyaluronidase Inhibitory Capacity of Fresh Jasmine Flower Extracts Obtained According to Examples 1 and 3

Hyaluronidase catalyzes the breakdown of hyaluronic acid (a glycosaminoglycan strongly presents in the dermis of the skin with moisturizing and anti-aging properties) into mono or disaccharides as well as smaller fragments of hyaluronic acid.

Hyaluronic acid has the ability to cause turbidity in the presence of an acidic albumin solution, which can be measured with a spectrophotometer at 600 nm.

Protocol: The enzyme is incubated with the extracts obtained according to Examples 1 and 3 at the chosen concentration (vol/vol dilution) at a temperature of 37° C. The enzyme's substrate, hyaluronic acid, is added to the mixture. The whole is slowly homogenized then left to incubate for exactly 45 minutes at 37° C. The hyaluronic acid remaining in the mixture is then contacted with an acidic albumin solution for 10 minutes before the transmittance is read at 600 nM on the spectrophotometer.

The extract of autofermented jasmine flowers prepared according to Example 1 has a significantly stronger enzyme inhibitory power than the extract prepared conventionally according to Example 3. The results are presented in [Table 4].

TABLE 4
Comparison of the percentages of hyaluronidase inhibition obtained,
in vitro, between conventional and autofermented jasmine extracts.

| | Conventional extract | | | Autofermented extract | | |
|---|---|---|---|---|---|---|
| Extract concentration | 0.5% | 1% | 2% | 0.5% | 1% | 2% |
| Hyluronidase inhibition | 4 | 1 | 15 | 57.4 | 86.6 | 100 |
| percentage | | | | | | |

### Example 7: Formula for a Rich Cream

TABLE 5

| Ingredients (Brand name) | INCI | % w/w |
|---|---|---|
| Phase A | | |
| Purified water | Aqua | Qsp 100 |
| Optiphen ™ Plus | Phenoxyethanol (and) Caprylyl | 1.50 |
| preservative | Glycol (and) Sorbic Acid | |
| Phase B | | |
| Stabileze ™ QM polymer | PVM/MA Decadiene Crosspolymer | 0.15 |
| Phase C | | |
| ProLipid ™ 141 lamellar | Glyceryl Stearate (and) | 5.00 |
| gel | Behenyl Alcohol | |
| | (and) Palmitic Acid (and) | |
| | Stearic Acid (and) Lecithin (and) | |
| | Lauryl Alcohol (and) | |
| | Myristyl Alcohol (and) | |
| | Cetyl Alcohol | |
| Ceraphyl ™ 494 ester | Isocetyl Stearate | 4.00 |
| Ceraphyl ™ SLK ester | Isodecyl Neopentanoate | 4.00 |
| DC 580 Wax | Stearoxytrimethylsilane (and) | 2.00 |
| | Stearyl Alcohol | |
| Emulsynt ™ GDL ester | Glyceryl Dilaurate | 3.00 |
| Phase D | | |
| Gransil DM-5 | Dimethicone (and) Polysilicone-11 | 3.00 |
| Phase E | | |
| Sodium hydroxyde | Sodium Hydroxide | 0.04 |
| Purified water | Aqua | 0.50 |
| Phase F | | |
| PF Precious wood | Perfume/Fragrance | 0.30 |
| Unipure* Red LC | CI 77491 (Iron oxides) (and) | 0.03 |
| 381 ADT-C | Isopropyl Titanium Triisostearate | |
| | (and) Bis-Hydroxyethoxypropyl | |
| | Dimethicone (and) | |
| | PEG-2-Soyamine (and) Isophorone | |
| | Diisocyanate | |
| Phase G | | |
| Extract according to | propanediol (and) Jasminum | 2.00 |
| example 2 | Grandiflorum Flower Extract | |
| Ronaflair Balance Gold | CI 77891 (Titanium | 0.30 |
| | Dioxide) (and) Mica | |
| | (and) Tin Oxide | |
| Covabead Velvet 10 | Polymethyl Methacrylate | 1.00 |
| Ronaflair Balance Red | CI 77891 (Titanium | 1.20 |
| | Dioxide) (and) Mica | |
| | (and) Tin Oxide | |
| Phase H | | |
| Purified water | Aqua | 15.00 |
| Natrosol ™ Plus 330 CS | Cetyl Hydroxyethylcellulose | 0.50 |
| HMHEC | | |

### Preparation Process

1. Homogenize phase A in the main container and start heating to 75-80° C.;
2. At 30° C., sprinkle in Phase B and homogenize while heating;
3. In a separate beaker, prepare phase C, heat to 75-80° C. until homogeneous;
4. At 75° C., add phase C to the main container and homogenize for 10 minutes;
5. Allow the temperature to cool and add phase D to 65° C. Mix well to homogenize for 10 minutes;
6. Premix phase E before adding it to the main container;
7. Add phase E at 60° C. Mix well to homogenize for 10 minutes;
8. At 35° C., premix phase F before adding it and mixing well;
9. Premix phase G before adding it to the main container;
10. Add phase G at 35° C. Mix well to homogenize;
11. In a separate beaker, prepare phase H: sprinkle Natrosol™ in water at room temperature and homogenize while heating to 60° C.;
12. Add phase H at 30° C. Mix well to homogenize;
13. Stop at 25° C.

The composition is thus in the form of a pink butter cream, with a pH between 4.90 and 5.40 and a viscosity (OD) of 160,000-210,000 cps (Brookfield RVT/Spindle D/5 RPM/1 minute/25° C.).

### Example 8: Evaluation of the Jasmine Extract Obtained According to Example 2 on the Expression of the Piezo 1 Receptor in Reconstructed Epidermis

The Piezo 1 and Piezo 2 proteins have been identified as ion channels mediating mechanosensory transduction in mammalian cells (Coste, Bertrand et al.) “Piezol and Piezo2 are essential components of distinct mechanically activated cation channels.” Science (New York. N.Y.) vol. 330.6000 (2010): 55-60). In the skin, keratinocytes help mediate tactile sensation by detecting and encoding this information for sensory neurons. Piezo1 is the main mechanotransducer of keratinocytes (Holt. Jesse R et al. Spatiotemporal dynamics of PIEZO1 localization controls keratinocyte migration during wound healing. eLife vol. 10 e65415. 27 Sep. 2021).

Principle: The jasmine extract obtained according to Example 2 was evaluated for its ability to modulate the expression of the Piezo 1 mechanoreceptor in human skin ex vivo.

Protocol: The expression of the Piezo 1 receptor is evaluated by indirect immunofluorescence on sections of reconstructed epidermis, previously treated by topical application of the jasmine extract from Example 2 diluted to 2% (vol/vol) for 48 hours (once a day). Reconstructed control epidermis incubated in parallel under the same conditions receive the placebo (Phosphate Butter Saline, PBS). At the end of the incubation, the reconstructed epidermis are fixed and embedded in paraffin for the production of histological sections. Detection of the Piezo 1 receptor is carried out by incubation with a primary anti-Piezo 1 antibody (Proteintech). After an hour and a half of incubation followed by rinsing, the sections are incubated in the presence of the anti-rabbit secondary antibody coupled to a fluorophore (Alexa Fluor® 488, Invitrogen). The sections are then examined under an Epifluorescence microscope (Zeiss Axiovert 200M microscope). The expression of the Piezo 1 receptor is then observed and quantified by image analysis (Vocity® image analysis software, Improvision).

Results: As illustrated by [FIG. 4], when the biopsies were treated with 2% jasmine extract, the expression of the Piezo 1 receptor increased by 39%.

Conclusion: Jasmine extract showed a positive effect on the expression of the Piezo 1 receptor.

### Example 9: Evaluation of the Jasmine Extract Obtained According to Example 2 on the Expression of the Oxytocin Receptor (OXTR) on Human Skin Biopsies

Principle: The aim of this experiment is to demonstrate an effect of jasmine extract on the synthesis of the oxytocin receptor in cultured human skin biopsies.

Protocol: The expression of the oxytocin receptor (OXTR) is evaluated by indirect immunofluorescence on skin biopsies, previously treated by topical application of the jasmine extract of Example 2 diluted to 2% (vol/vol) or 48 hours (twice a day). Control biopsies incubated in parallel under the same conditions receive the placebo (Phosphate Butter Saline, PBS). At the end of the incubation, the biopsies are fixed and embedded in paraffin for the production of histological sections. Detection of the OXTR receptor is carried out by incubation with the anti-OXTR antibody (Proteintech). After an hour and a half of incubation followed by rinsing, the sections are incubated in the presence of the anti-rabbit secondary antibody coupled to a fluorophore (Alexa Fluor® 488, Invitrogen). The sections are then examined under an Epifluorescence microscope (Zeiss Axiovert 200M microscope). The expression of the OXTR receptor is then observed and quantified by image analysis (Vocity® image analysis software, Improvision).

Results: As illustrated by [FIG. 5], when the biopsies were treated with 2% jasmine extract, the expression of the OXTR receptor increased by 63%.

Conclusion: Jasmine extract showed a positive effect on the expression of the olfactory oxytocin receptor OXTR.

### Example 10: Evaluation of the Jasmine Extract Obtained According to Example 2 on the Expression of Oxytocin Receptor (OXTR) Messenger RNAs in Cultured Keratinocytes

Oxytocin is a natural peptide controlling a wide range of specific actions in its target tissues, ranging from cell growth and differentiation to reproduction and social behavior (Carter, C Sue et al. “Is Oxytocin “Nature's Medicine Pharmacological reviews vol. 72.4 (2020): 829-861). Many scientific publications support the beneficial role of oxytocin in skin physiology and aging (Hayre, Nicole. “Oxytocin Levels Inversely Correlate with Skin Age Score and Solar Damage.” Journal of drugs in dermatology: JDD vol. 19.12 (2020): 1146-1148). Other research also highlights the importance of the oxytocin receptor in preventing skin aging (Cho, S-Y et al. “Oxytocin alleviates cellular senescence through oxytocin receptor-mediated extracellular signal-regulated kinase/Nrf2 signaling.” The British journal of dermatology vol. 181.6 (2019): 1216-1225).

Principle: The aim of this experiment is to demonstrate an effect of jasmine extract on the synthesis of oxytocin receptor messenger RNA in human skin keratinocytes.

Protocol: The level of expression of OXTR oxytocin receptor messenger RNA is evaluated by qPCR (quantitative polymerase chain reaction) on skin keratinocytes in culture, previously treated with jasmine extract obtained according to the Example 2 for 48 hours at 1% (vol/vol dilution) in the culture medium (once per day). At the end of the culture, the cells are lysed, the total RNA is extracted then converted into complementary DNA (cDNA) by a reverse transcription reaction. Quantification of OXTR receptor cDNAs was performed by qPCR using a TaqMan probe for detection. The delta-delta Ct quantification method made it possible to compare the difference in expression (ΔCt) between the gene of interest (OXTR) and the reference gene (GADPH).

Results: As illustrated by [FIG. 6], when the keratinocytes in culture were treated with the jasmine extract obtained in Example 2 at 1%, the expression of messenger RNAs of the OXTR receptor was increased by 26%.

Conclusion: Jasmine extract showed a positive effect on the expression of OXTR receptor messenger RNAs.

### Example 15: Evaluation of the Jasmine Extract Obtained According to Example 2 on the Expression of Viperin in Cultured Keratinocytes

Viperin is one of the innate immune defense factors synthesized by keratinocytes (Garcia. Magali et al. “Innate Immune Response of Primary Human Keratinocytes to West Nile Virus Infection and Its Modulation by Mosquito Saliva.” Frontiers in cellular and infection microbiology vol. 8 387. 2 Nov. 2018.). Viperin has been shown to catalyze the conversion of cytidine triphosphate (CTP) to 3′-deoxy-3′,4′-didehydro-CTP (ddhCTP), a previously unknown ribonucleotide analogue. Incorporation of ddhCTP causes premature termination of RNA synthesis in certain viruses (Gizzi. Anthony S et al. “A naturally occurring antiviral ribonucleotide encoded by the human genome.” Nature vol. 558.7711 (2018): 610-614.).

Principle: The aim of this experiment is to demonstrate an effect of jasmine extract on the level of expression of viperin in cultured keratinocytes.

Protocol: Viperin expression is evaluated by indirect immunofluorescence on keratinocytes. The cultured keratinocytes were treated by application of the jasmine extract obtained according to example 2 at 1% (vol/vol dilution) for 48 hours (once a day). At the end of the culture, the cells are fixed. Detection of viperin is carried out by incubation with an antiviperin antibody (Sigma). After an hour and a half of incubation followed by rinsing, the cells are incubated in the presence of an anti-mouse secondary antibody coupled to a fluorophore (Alexa Fluor® 488. Invitrogen). The cells are then examined under an Epi-fluorescence microscope (Zeiss Axiovert 200M microscope). The expression of viperin is then observed and quantified by image analysis (Volocity® image analysis software. Improvision).

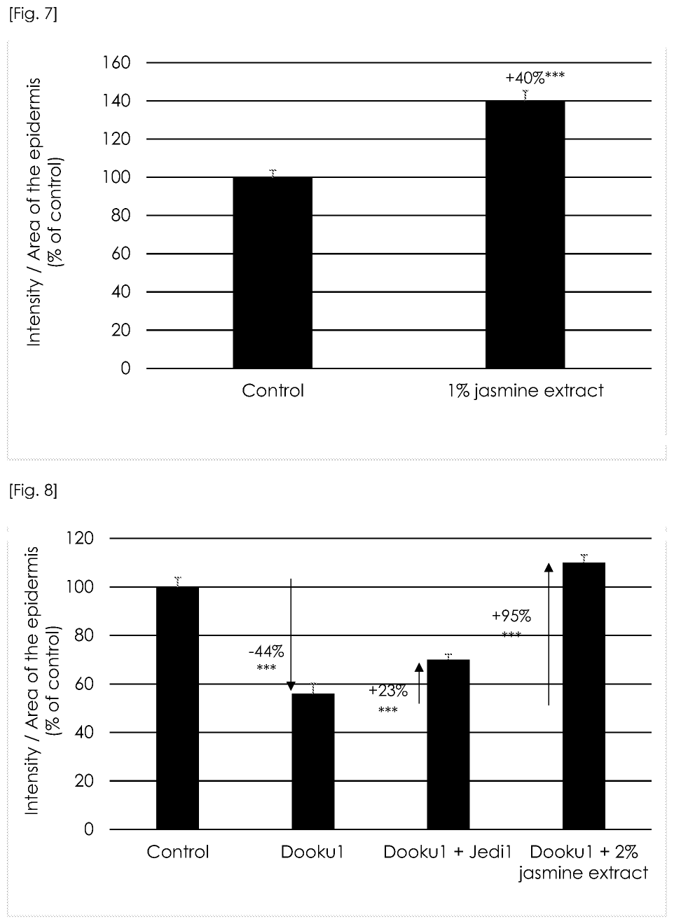

Results: As illustrated by [FIG. 7], the jasmine extract obtained according to example 2 showed a positive effect of +40% on the expression of viperin in cultured keratinocytes.

Conclusion: Jasmine extract showed a positive effect on viperin expression.

### Example 16: Evaluation of the Jasmine Extract Obtained According to Example 2 on the Level of Expression of E-Cadherin, on Human Skin Biopsies Pretreated with a Blocker of Piezo 1 Activity (Dooku 1)

Principle: The aim of this experiment is to demonstrate an effect of jasmine extract on the level of expression of E-cadherin, a molecule involved in tissue tone, in human skin biopsies in culture.

Protocol: The expression of E-cadherin is evaluated by indirect immunofluorescence on skin biopsies pretreated with a blocker of Piezo 1 activity (Dooku 1), then treated by topical application of jasmine extract obtained according to the example 2 for 48 hours (twice a day), or with a Piezo 1 activator (Jedi 1) for 48 hours (once a day). Control biopsies incubated in parallel under the same conditions receive the placebo (Phosphate Butter Saline, PBS). At the end of the incubation, the biopsies are fixed and embedded in paraffin for making histological sections. Detection of E-cadherin is carried out by incubation with anti-E-cadherin antibodies (Abcam). After an hour and a half of incubation, followed by rinsing, the sections are incubated in the presence of the anti-rabbit secondary antibody coupled to a fluorophore (Alexa Fluor® 488. Invitrogen). The sections are then examined under an Epi-fluorescence microscope (Zeiss Axiovert 200M microscope). The expression of the OXTR receptor is then observed and quantified by image analysis (Volocity® image analysis software. Improvision).

Results: As illustrated by [FIG. 8], when the biopsies were pretreated with a Piezo 1 activity blocker (Dooku 1), α—44% decrease in E-cadherin expression was observed. α—44% decrease in E-cadherin expression was observed. When the biopsies were treated with Piezo lactivator (Jedi 1), the expression of E-cadherin was increased by 23%. When the biopsies were treated with jasmine extract, the expression of E-cadherin was increased by 95%.

Conclusion: The jasmine extract obtained according to example 2 showed a positive effect on the expression of E-cadherin, by mimicking the effect of the Piezo 1 receptor activator (Jedi 1).

### Example 17: Evaluation of the Jasmine Extract Obtained According to Example 2 on the Level of Expression of the Messenger RNAs of the Enzyme 11ß-HSD1 in Cultured Keratinocytes

Principle: The aim of this experiment is to demonstrate an effect of jasmine extract on the level of expression of the messenger RNA of the enzyme 11β-HSD1, also known as cortisone reductase, in Human skin keratinocytes in culture. An increase in the expression of the enzyme 11β-HSD1 has been implicated in the degradation of epidermal barrier function during skin aging (Kim. B. J. et al. Increased Expression of 11-Hydroxysteroid Dehydrogenase Type 1 Contributes to Epidermal Permeability Barrier Dysfunction in Aged Skin. Int. J. Mol. Sci. 2021. 22. 5750.).

Protocol: The level of expression of messenger RNAs of the enzyme 11β-HSD1 is evaluated by qPCR (quantitative polymerase chain reaction) on human skin keratinocytes in culture, previously treated with jasmine extract obtained according to Example 2 for 48 hours at 1% in the culture medium (once a day). At the end of the culture, the cells are lysed, the total RNA is extracted then converted into complementary DNA (cDNA) by a reverse transcription reaction. Quantification of enzyme 11β-HSD1 CDNAs was carried out by quantitative chain polymerization using a TaqMan probe for detection. The delta-delta Ct quantification method made it possible to compare the difference in expression (ΔCt) between the gene of interest (11β-HSD1) and the reference gene (GADPH).

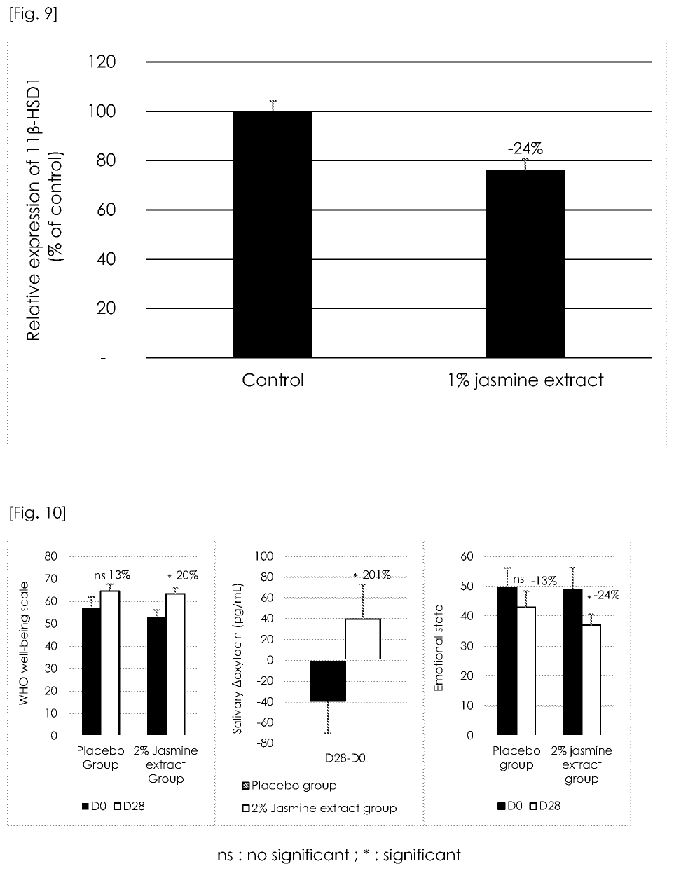

Results: As illustrated by [FIG. 9], when the keratinocytes in culture were treated with the jasmine extract obtained according to example 2 at 1%, the level of expression of the mRNA of the 11β-HSD1 enzyme was reduced by −24%.

Conclusion: Jasmine extract showed an inhibition effect on the expression level of the 11β-HSD1 enzyme.

### Example 18: Evaluation of the Jasmine Extract Obtained According to Example 2 to Combat the Signs of Skin Aging, Improve the Barrier Function, Luminosity of the Skin and Well-Being In Vivo

Principle: Study the effectiveness of Jasmine extract formulated at 2% obtained

Protocol: comparative study carried out double-blind against a placebo, carried out on the face and forearm of 34 volunteers (aged 36 to 66 years) divided into two groups of 17 volunteers homogeneous according to age and sex. The treated group received the Jasmine extract from Example 2 formulated at 2% in the skin care formula detailed in Table 6 and the placebo group received the same care formula but not containing the jasmine extract. Product allocation was carried out according to a randomization list created by a random number generator.

TABLE 6
Care formula containing the Jasmine extract from example 2 at 2% used
during the clinical test

| ingredients (trade name \| INCI) | | % w/w | supplier |
|---|---|---|---|
| phase A | | | |
| Purified water | Water/Aqua | qs. | Local |
| | | 100 | |
| EDTA tetrasodium Salt | Tetrasodium EDTA | 0.05 | Fisher |
| Lubrajel* MS Free | Glycerin (and) Glyceryl | 3.00 | Ashland |
| hydrogel | Acrylate/Acrylic Acid | | |
| | Copolymer (and) | | |
| | Phenoxyethanol | | |
| LiquaPar ™/Rokonsal ™ | Phenoxyethanol (and) | 1.00 | Ashland |
| MEP preservative | Methylparaben (and) | | |
| | Ethylparaben (and) | | |
| | Propylparaben | | |
| phase B | | | |
| UltraThix ™ P-100 | Acrylic Acid/VP | 0.60 | Ashland |
| polymer | Crosspolymer | | |
| phase C | | | |
| Sodium Hydroxide | Sodium Hydroxide | 0.02 | Fisher |
| Purified water | Water/Aqua | 0.50 | Local |
| phase D | | | |
| Belsil* W3230 | Bis-Stearoxydimethylsilane | 2.00 | Wacker |
| | (and) Stearyl Alcohol (and) | | |
| | Dimethicone | | |
| Simulsol* 165 | PEG-100 Stearate (and) | 2.00 | Seppic |
| | Glyceryl Stearate | | |
| Refined Shea Butter | Butyrospermum Parkii | 2.00 | Ashland |
| | (Shea) Butter | | |
| Ceraphyl ™ 28 ester | Cetyl Lactate | 1.50 | Ashland |
| Ceraphyl ™ 791 ester | Isocetyl Stearoyl Stearate | 2.00 | Ashland |
| Ceraphyl ™ ODS ester | Octyldodecyl Stearate | 3.00 | Ashland |
| Ceraphyl ™ 368 ester | Ethylhexyl Palmitate | 4.00 | Ashland |
| phase E | | | |
| Sodium Hydroxide | Sodium Hydroxide | 0.03 | Fisher |
| Purified water | Water/Aqua | 0.50 | Local |
| Phase F | | | |
| Jasmine extract | | 2.0 | Ashland |
| according example 2 | | | |
| total | | 100.00% | |

Duration of the study: 28 days including 3 control visits at DO (first day of the study), D14 (after 14 days of applications) and D28 (end of the study after 28 days of applications).
Measurement of well-being by three different methods at DO and D28:
   a. Anonymized questionnaire on the well-being index designed by the WHO.
   b. Quantification of oxytocin in saliva by ELISA (enzyme linked immunosorbent assay) method. Oxytocin is a nonapeptide that plays a key role in several physiological functions (e.g., milk ejection) as well as in certain types of behavioral regulation such as attachment, social recognition, and anxiety. Several studies suggest that pleasant touch is associated with the release of oxytocin and induces relaxing behavioral effects After rinsing the mouth thoroughly for 30 seconds, 2 ml of volunteers' saliva was collected in a pre-inflated tube and immediately stored at −80° C. Salivary oxytocin was quantified using an ELISA kit (Enzo Life Science).
   c. Measurement of emotional state using the Emotion Capture® system patented by the company ICONIK®. This biometric technology measures a person's state of stress in real time, using 3 different sensors measuring:
      i. changes in the ionic charge of the sweat gland by skin-sensitive galvanic sensor
      ii. cardiac modifications by optical sensor
      iii. skin temperature changes by skin temperature sensor:
         All the data collected is then normalized and processed by an algorithm developed by ICONIK, the emotional state of the volunteers and their stress rate is calculated.
Insensible water loss (TEWL)—on the forearm measured by a closed chamber method using the AquaFlux® AF200 (Biox®)
Skin tone-on the face measured with the CM-700d spectrophotometer CM-700d (Konica Minolta Sensing. Inc.). The ITA° (Individual Topology Angle) parameter was calculated with the following equation ITA=(Arc tan L*−50/b*)×180/π).
L*: skin luminance value
b*: values between blue and yellow (negative values indicate blue and positive values indicate yellow)
Topography of the skin of the forehead by silicone replica, analyzed by the combination of fringe projection and stereometry technologies with the AEAVA-HER system (Eotech®). Different parameters of the skin microrelief were calculated, such as the surface and profile roughness parameters. In addition, 2D and 3D representations of the topography of the skin could be produced with this same system.
Color photos of the face. The photos were taken with the HeadScan*V03 bench (Orion Concept*).
Evaluation on a scale of 0 to 100 by an expert of the condition of the facial skin to the touch and according to the visual appearance of the following parameters:
Skin tone: 0 corresponding to very dull skin and 100 to perfect luminosity and complexion.
Homogeneity of the skin: 0 corresponding to non-homogeneous skin with redness and spots and 100 to very homogeneous skin without redness and spots.
Skin hydration: 0 corresponding to very dry skin and 100 to very hydrated skin.
Crow's feet fine lines: 0 corresponding to smooth skin without fine lines and 100 to skin with marked microrelief and visible fine lines.
Overall appearance of the skin: 0 corresponding to poor hydration of the skin, very rough, without luminosity and non-homogeneous and 100 to very hydrated, luminous, homogeneous, and smooth skin.

All measurements were carried out on the subject after a 15-minute rest period in a room where the temperature (21° C.+/−1) and relative humidity (50%+/−5) were controlled.

Well-being measurement results: as illustrated in [FIG. 10], the results show that after one month of application, the volunteers in the treated group improved their well-being. Analysis of the WHO Well-being Index questionnaire shows a 20% increase in felt well-being compared to DO for the treated group, which is not the case for the placebo group. There was also a significant increase in oxytocin in saliva in the treated group compared to the placebo group, suggesting that the application of jasmine extract may help improve oxytocin release. Finally, the treated group showed a significant improvement in their emotional state between D0 and D28, with the volunteers feeling less stressed, while no significant improvement was recorded in the placebo group.

Conclusions from well-being measurements: these results demonstrate that Jasmine extract formulated at 2% can improve well-being in-vivo.

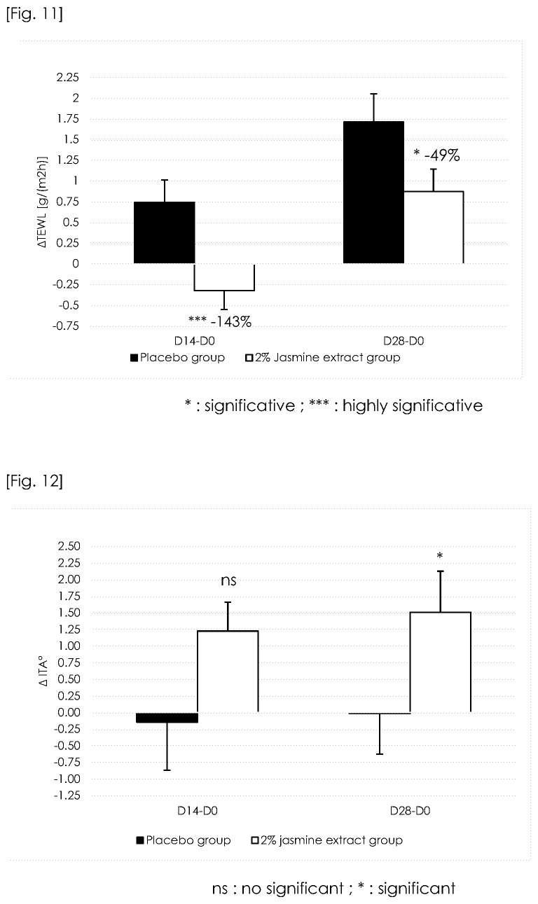

Result of measurements of insensible water loss (TEWL): As shown in [FIG. 11], the imperceptible loss of water is significantly lower for the treated group than for the placebo group from 14 days of application until the end of the study.

Conclusion of the measurements of insensible water loss: this result demonstrates that the skin barrier has been strengthened after 14 days of applications of Jasmine extract formulated at 2%.

Results of skin tone measurements: [FIG. 12] shows that after one month of application of the cream containing 2% jasmine extract, the ITA° increases significantly compared to placebo.

Conclusion of skin tone measurements: this result demonstrates that jasmine extract formulated at 2% helps improve facial complexion.

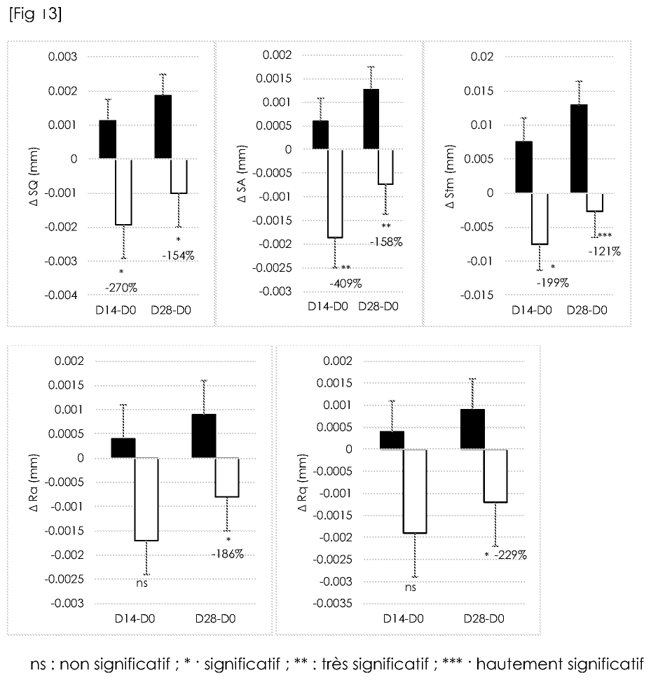

Results of forehead skin topography measurements and facial color photos: as shown in [FIG. 13], the surface roughness and profile parameters at the forehead decreased for the treated group compared to the placebo group from 14 days of application. In addition, smoother skin and less visible microrelief could be observed in the 3D photos of volunteers in the treated group, unlike volunteers in the placebo group. These results are confirmed by observation of color photos of the face.

Conclusion of the measurements of the topography of the skin of the forehead and the color photos of the face: these results highlight an anti-aging effect of jasmine extract formulated at 2%.

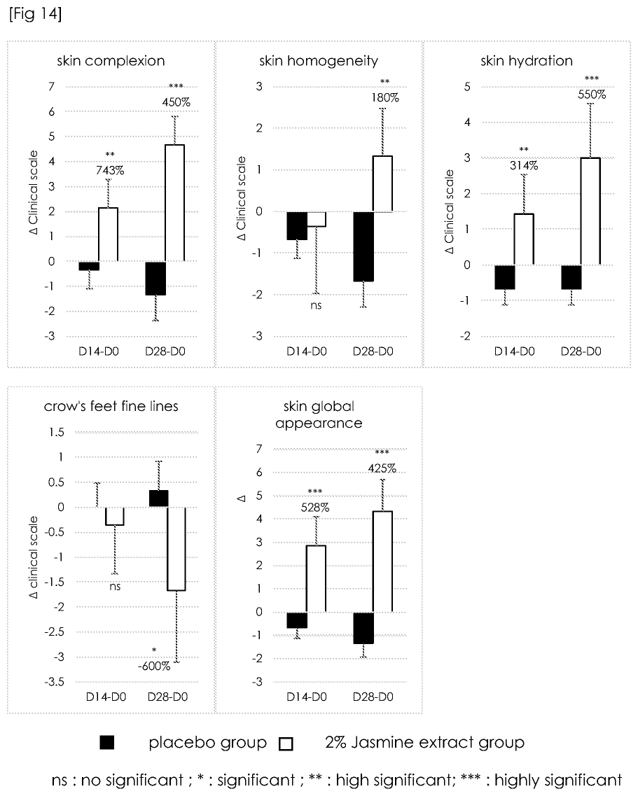

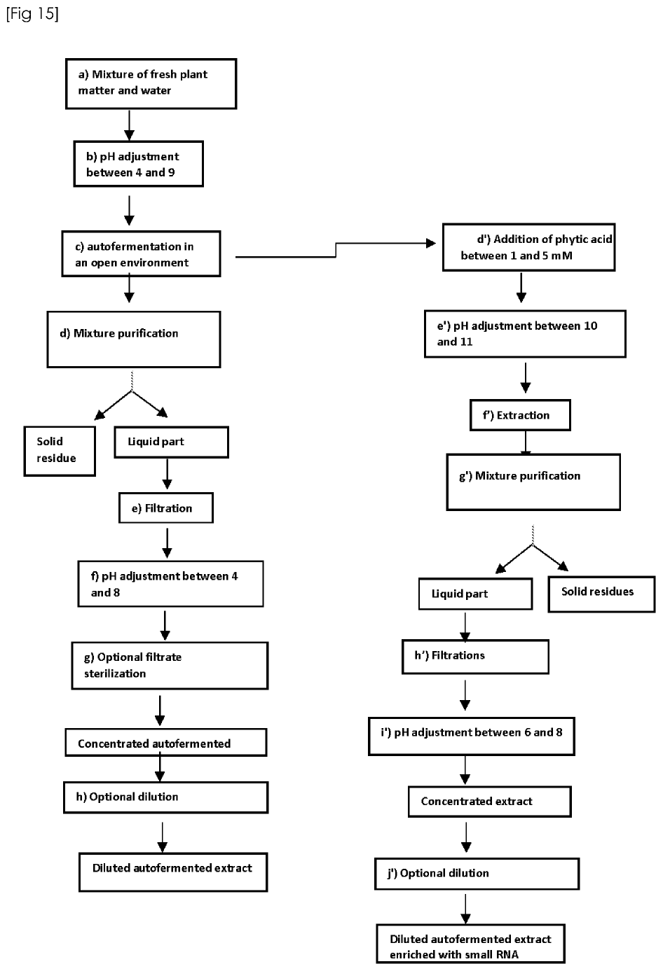

Results of expert assessment: according to [FIG. 14], after 14 days of application with 2% Jasmine extract, the expert observed an improvement in complexion, hydration and overall appearance of the skin, and no improvement after 14 days of application of the placebo. Likewise, after one month of application, the expert noticed an improvement in all the parameters evaluated in the volunteers in the treated group and not in the volunteers in the placebo group.

Conclusion of the evaluation by an expert: these results demonstrate that Jasmine extract formulated at 2% makes it possible to visually reduce facial fine lines as well as improve facial hydration and complexion.

Conclusion of the test: under current conditions, the results above demonstrate that Jasmine extract formulated at 2% improves the well-being of volunteers and reduces the effects of skin aging.

What is claimed is:

1. A process for obtaining plant extracts enriched in organic acids and phenolic acids, comprising the following steps:
a) placing the plant material into water;
b) if necessary, adjusting the pH to a value comprised between 4 and 9;
c) keeping the mixture under gentle stirring for a time comprised between 6 and 48 hours, at a temperature comprised between 2° and 60° C., at a pH comprised between 4 and 7, to allow the autofermentation process to take place, in an enclosure allowing gas exchange with the atmosphere;
d) purifying the mixture obtained in c) to eliminate residual solid plant matter and collecting the liquid part;
e) filtering at least once the liquid obtained in the previous step;
f) checking the pH of the filtrate and readjusting it, if necessary, to a value comprised between 4 and 8, and preferably comprised between 4 and 7;
g) sterilizing the filtrate;
h) diluting the extract with a physiologically acceptable solvent to obtain diluted plant extracts characterized in that they comprise, by weight of the total weight of the extract, from 2 to 20 g/kg of dry extract, from 0.2 to 4 g/kg of sugars, from 100 to 700 mg/kg of organic acids including 50 to 350 mg/Kg of shikimic acid and from 10 to 70 mg/Kg of quinic acid, from 100 to 1300 mg/kg of phenolic compounds including 70 to 140 mg/Kg of phenolic acids and from 0.1 to 5 g/Kg of proteins.

2. The process for preparing plant extracts according to claim 1, characterized in that step c) of autofermentation takes place for a time comprised between 15 and 30 h, at a temperature comprised between 30 and 40° C.

3. The process for preparing plant extracts, characterized in that it comprises steps a) to c) according to claim 1, followed by the following subsequent steps:
Step d′) adding phytic acid to the mixture obtained in step c) at a concentration comprised between 1 and 5 mM;
Step e′) if necessary, adjusting the pH to a value comprised between 10 and 11,
Step f′) keeping the mixture under stirring for 1 to 3 hours, at a temperature comprised between 50° C. and 80° C.,
Step g′) removing the residual plant material,
Step h′) purifying the obtained liquid fraction by successive filtrations to clarify the extract,
Step i′) adjusting the pH of the resulting mixture to a value comprised between 6 and 8,
Step j′) Optionally, diluting the extract with a physiologically acceptable solvent.

4. The process for preparing plant extracts according to claim 1, characterized in that the plant materials used are plants of the oleaceae, poaceae, or fabaceae family.

5. The process for preparing plant extracts according to claim 1, characterized in that the plant material used is fresh jasmine flower, preferably of the species Jasminum grandiflorum.

6. A diluted plant extracts obtained by the process of claim 3, characterized in that they comprise at least 300 mg/kg of phenolic compounds including at least 140 mg/Kg of phenolic acids and at least 300 mg/kg of organic acids, including at least 50 mg/Kg of shikimic acid and 10 mg/kg of quinic acid.

7. The diluted plant extracts obtained by the process of claim 3, characterized in that they comprise, by weight of the total weight of the extract, from 2 to 40 g/kg dry weight; 0.2 to 5 g/kg sugars; 300 to 900 mg/kg of organic acids including 50 to 500 mg/Kg of shikimic acid and 10 to 200 mg/kg of quinic acid; 300 to 2000 mg/kg of phenolic compounds including 140 to 210 mg/Kg of phenolic acids and 2 to 10 g/kg of proteins, as well as 10 to 60 mg/Kg of small molecular weight RNA.

8. The diluted plant extracts obtained by the process of claim 3, characterized in that the plant material used is fresh jasmine flower, preferably of the species Jasminum grandiflorum, and in that it further comprises at least one jasmintide.

9. A composition comprising an effective amount of the extract obtained according to the process of claim 1, preferably comprising from 0.05 to 5% of the said extract, and even more preferably from 0.1 to 2.5% of the said extract by weight relative to the total weight of the composition, and a physiologically acceptable medium.

10. A cosmetic method of treatment comprising applying the composition of claim 9 to the skin to combat signs of skin ageing, the loss of firmness or tone, to improve barrier function and hydration, brighten the skin or improve the skin's innate immune defenses and in vivo well-being.

11. A cosmetic method of treatment comprising applying the composition of claim 9 to the skin to alleviate the age-related decrease in touch receptors (piezo 1) and oxytocin receptors (OXTR).

12. (canceled)

13. The process for preparing plant extracts according to claim 3, characterized in that the plant materials used are plants of the oleaceae, poaceae, or fabaceae family.

14. A composition comprising an effective amount of the extract obtained according to the process of claim 3, preferably comprising from 0.05 to 5% of the said extract, and even more preferably from 0.1 to 2.5% of the said extract by weight relative to the total weight of the composition, and a physiologically acceptable medium.

15. A cosmetic method of treatment comprising applying the composition of claim 13 to the skin to combat signs of skin ageing, the loss of firmness or tone, to improve barrier function and hydration, brighten the skin or improve the skin's innate immune defenses and in vivo well-being.

16. A cosmetic method of treatment comprising applying the composition of claim 13 to the skin to alleviate the age-related decrease in touch receptors (piezo 1) and oxytocin receptors (OXTR).
